SEQUENCE LISTING (3 sequences)

SEQ ID NO: 1 - protein, 164 aa, Burkholderia cenocepacia
IGLNGGANVYHYAPNPVGWVDPWGLAGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIPVKRGATGETKVFTGNSNSPKSPTKGGC

SEQ ID NO: 2 - protein, 151 aa, Pseudomonas syringae
KVSNIAESEAALGRASQARADLPQSKELKVKTVSSNDKKTLSGWGNKKPEGYERISAEQVKAKSEEIGHEVKSHPYDRDYKGQYFSSHAEKQMSIASPNHPLGVSKPMCTDCQGYFSQLAKYSKVEQTVADPKAIRIFKTDGSVETIMRSE

SEQ ID NO: 3 - DNA, 35 bp, Artificial sequence
  Synthetic
aaaaaaaaaaaaaagctcggaaaaaaaaaaaaaaa

(19) United States
(12) Patent Application Publication
(10) Pub. No.: US 2024/0124867 A1
(43) Pub. Date: Apr. 18, 2024

(54) BACTERIAL DNA CYTOSINE DEAMINASES FOR MAPPING DNA METHYLATION SITES

(71) Applicant: University of Washington, Seattle, WA (US)

(72) Inventors: Joseph D. Mougous, Seattle, WA (US); Jay Ashok Shendure, Seattle, WA (US); Jean-Benoît Lalanne, Seattle, WA (US); Snow Brook Peterson, Seattle, WA (US); Larry A. Gallagher, Seattle, WA (US)

(73) Assignee: University of Washington, Seattle, WA (US)

(21) Appl. No.: 18/547,629

(22) PCT Filed: Mar. 30, 2022
(86) PCT No.: PCT/US2022/022655

§ 371 Date: Aug. 23, 2023

Related U.S. Application Data
(60) Provisional application No. 63/169,425, filed on Apr. 1, 2021.

(51) Int. Cl. C12N 9/78; C12N 15/10; C12Q 1/34

(52) U.S. Cl. CPC C12N 9/78; C12N 15/1003; C12Q 1/34; C12Y 305/04001

(57) ABSTRACT

The disclosure provides methods and related kits, reagents, and systems for selectively deaminating unmethylated cytosine residues in nucleic acid molecules. In some embodiments, the methods and related kits, reagents, and systems are applied for methods of detecting and/or mapping methylated cytosine residues in nucleic acids. The nucleic can be RNA or DNA. Some embodiments include contacting the polynucleic acid with a bacterial cytosine deaminase, for example DddA or SsdA, or functional fragments or derivatives thereof. Representative DddA and SsdA have sequences set forth in SEQ ID NOS:1 and 2, respectively. The bacterial cytosine deaminases of the disclosure are sensitive to methylation and, thus, deaminate only unmethylated cytosines to provide a cytosine to uracil conversion. The conversion can be detected as a C•G-to-T•A transitions in subsequent sequencing analysis.

### CROSS-REFERENCE TO RELATED APPLICATION

This application claims the priority benefit of U.S. Provisional Application No. 63/169,425, filed Jul. 10, 2019, which is incorporated herein by reference in its entirety for all purposes.

### STATEMENT REGARDING SEQUENCE LISTING

The sequence listing associated with this application is provided in text format in lieu of a paper copy and is hereby incorporated by reference into the specification. The name of the text file containing the sequence listing is 3915_P1199WOUW_Seq_List_FINAL_20220329_ST25.txt. The text file is 4 KB; was created on Mar. 29, 2022; and is being submitted via EFS-Web with the filing of the specification.

### BACKGROUND

Methylation of cytosine residues in DNA is an important component of epigenetic gene regulation in many eukaryotic organisms. In addition, methylation status of particular chromosomal sites has emerged as a key diagnostic biomarker for a number of cancers. However, the of current technologies available for detecting sites of cytosine methylation in DNA have limitations, including significant template loss or degradation of template, multiple chemical or enzymatic treatments, specific reaction conditions, harsh chemical treatments, specialized lab equipment, and the like. These limitations have prevented the widespread implementation of methylation-based diagnostics.

Accordingly, there remains a need in the art for an efficient, facile, sensitive, and accurate approach to detect methylation of cytosine residues in DNA. The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a method of deaminating one or more unmethylated cytosine residues in a polynucleic acid molecule. The method comprises contacting the polynucleic acid molecule with a bacterial cytosine deaminase.

In some embodiments, the bacterial cytosine deaminase does not deaminate methylated cytosines in the polynucleic acid.

In some embodiments, the bacterial cytosine deaminase is double-stranded DNA deaminase toxin A (DddA), or a functional fragment or derivative thereof. In some embodiments, the DddA or functional fragment or derivative of DddA comprises an amino acid sequence with at least 130 contiguous amino acids of SEQ ID NO:1 or an amino acid sequence with at least about 80% identity to 130 contiguous amino acids of SEQ ID NO:1. In some embodiments, the DddA or functional derivative or derivative of DddA comprises an amino acid sequence with at least about 80%, at least about 85%, at least about 90%, at least about 95%, or at least about 98% to the amino acid sequence of SEQ ID NO:1. In some embodiments, the DddA or functional fragment or derivative thereof is contacted to the polynucleic acid molecule in a reaction wherein the functional fragment or derivative thereof is present at a concentration of about 0.5 nM to about 10 nM.

In some embodiments, the bacterial cytosine deaminase is single-stranded DNA deaminase toxin A (SsdA), or a functional fragment or derivative thereof. In some embodiments, the SsdA or a functional fragment or derivative of SsdA comprises an amino acid sequence with at least 130 contiguous amino acids of SEQ ID NO:2 or an amino acid sequence with at least about 80% identity to 130 contiguous amino acids of SEQ ID NO:2. In some embodiments, the SsdA or a functional fragment or derivative of SsdA comprises an amino acid sequence with at least about 80%, at least about 85%, at least about 90%, at least about 95%, or at least about 98% to the amino acid sequence of SEQ ID NO:2.

In some embodiments, the method further comprises isolating or purifying the polynucleic acid from a biological sample. In some embodiments, the polynucleic acid is DNA. In some embodiments, the DNA is genomic or mitochondrial DNA. In some embodiments, the method further comprises isolating the DNA from a cell or plurality of cells.

In some embodiments, deamination of the one or more cytosine residues in the polynucleic acid molecule results in a cytosine to uracil conversion. In some embodiments, the method further comprises detecting the occurrence of one or more deamination events in the polynucleic acid. In some embodiments, detecting the occurrence of the deamination event(s) in the polynucleic acid comprises sequencing the polynucleic acid after contacting with the bacterial cytosine deaminase and detecting introduction of one or more C•G-to-T•A transitions in the polynucleic acid. In some embodiments, detecting introduction of one or more CG to TA transitions in the polynucleic acid comprises comparing the sequence of the polynucleic acid with a reference polynucleic acid sequence obtained from a reference polynucleic acid that has not been contacted with the bacterial cytosine deaminase. In some embodiments, the reference polynucleic acid is obtained from the same or similar biological sample as the polynucleic acid molecule contacted with the bacterial cytosine deaminase.

In another aspect, the disclosure provides a method of mapping methylated cytosine residues in a polynucleic acid molecule. The method comprises:
contacting a target polynucleic acid molecule with a bacterial cytosine deaminase for a sufficient time to deaminate unmethylated cytosine residues in the polynucleic acid molecule to provide a treated polynucleic acid molecule;
sequencing the treated polynucleic acid molecule to provide a treated sequence;
comparing the treated sequence to a reference sequence obtained from a reference polynucleic acid molecule identical to the target polynucleic acid molecule, wherein the reference polynucleic acid molecule is not contacted with a bacterial cytosine deaminase;
detecting introduction of one or more CG to TA transitions in the treated sequence compared to the reference sequence. The one or more CG to TA transitions correspond to unmethylated cytosine residues in the target polynucleotide and/or C residues in the treated sequence correspond to methylated cytosine residues in the target polynucleotide.

In some embodiments, the bacterial cytosine deaminase is double-stranded DNA deaminase toxin A (DddA), or a functional fragment or derivative thereof. In some embodiments, the DddA or functional fragment or derivative of DddA comprises an amino acid sequence with at least 130 contiguous amino acids of SEQ ID NO:1 or an amino acid sequence with at least about 80% identity to 130 contiguous amino acids of SEQ ID NO:1. In some embodiments, the DddA or functional derivative or derivative of DddA comprises an amino acid sequence with at least about 80%, at least about 85%, at least about 90%, at least about 95%, or at least about 98% to the amino acid sequence of SEQ ID NO:1. In some embodiments, the DddA or functional fragment or derivative thereof is contacted to the polynucleic acid molecule in a reaction wherein the functional fragment or derivative thereof is present at a concentration of about 0.5 nM to about 10 nM.

In some embodiments, the bacterial cytosine deaminase is SsdA, or a functional fragment or derivative thereof. In some embodiments, the SsdA or a functional fragment or derivative of SsdA comprises an amino acid sequence with at least 130 contiguous amino acids of SEQ ID NO:2 or an amino acid sequence with at least about 80% identity to 130 contiguous amino acids of SEQ ID NO:2. In some embodiments, the SsdA or a functional fragment or derivative of SsdA comprises an amino acid sequence with at least about 80%, at least about 85%, at least about 90%, at least about 95%, or at least about 98% to the amino acid sequence of SEQ ID NO:2.

In some embodiments, the polynucleic acid is DNA. In some embodiments, the DNA is genomic or mitochondrial DNA. In some embodiments, the method further comprises isolating the DNA from a biological sample.

In another aspect, the disclosure provides a kit comprising a bacterial cytosine deaminase and reagents configured to facilitate deamination of cytosine residues in a polynucleic acid.

In some embodiments, the bacterial cytosine deaminase is DddA, or a functional fragment or derivative thereof. In some embodiments, the DddA or functional fragment or derivative of DddA comprises an amino acid sequence with at least 130 contiguous amino acids of SEQ ID NO:1 or an amino acid sequence with at least about 80% identity to 75 contiguous amino acids of SEQ ID NO:1. In some embodiments, the DddA or functional derivative or derivative of DddA comprises an amino acid sequence with at least about 80%, at least about 85%, at least about 90%, at least about 95%, or at least about 98% to the amino acid sequence of SEQ ID NO:1.

In some embodiments, the bacterial cytosine deaminase is SsdA, or a functional fragment or derivative thereof. In some embodiments, the SsdA or a functional fragment or derivative of SsdA comprises an amino acid sequence with at least 130 contiguous amino acids of SEQ ID NO:2 or an amino acid sequence with at least about 80% identity to 75 contiguous amino acids of SEQ ID NO:2. In some embodiments, the SsdA or a functional fragment or derivative of SsdA comprises an amino acid sequence with at least about 80%, at least about 85%, at least about 90%, at least about 95%, or at least about 98% to the amino acid sequence of SEQ ID NO:2.

In some embodiments, the reagents configured to facilitate deamination comprise one or more of buffers, salts, and the like. In some embodiments, the reagents configured to facilitate deamination comprise a deamination buffer comprising NaCl, MES, DTT, and/or Ficoll PM70.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1D. Comparison of a DddA-based technique to established methods for defining DNA methylation sites. 1A) Traditional method of detecting methylated cytosines through bisulfite conversion followed by sequencing. Substrate degradation leads to significant sample loss. 1B) Enzymatic method for methylation detection (EM-Seq), requiring two enzymatic treatments prior to sequencing. 1C) TAPS method for methylation mapping through enzymatic cytosine oxidation followed by chemical conversion to dihydrouracil (DHU) and sequencing. 1D) DddA- (or other bacterial deaminase) based methylation site mapping requires a single enzymatic treatment that maintains sample integrity, followed by sequencing.

FIGS. 2A-2C. Activity of bacterial cytosine deaminases DddA (2A) and SsdA (2B, 2C) is blocked by cytosine methylation. 2A) A double stranded oligonucleotide (S; GTCGG) containing unmethylated (left) or methylated cytosine (right) was treated with the indicated concentration of DddA. Deamination of cytosine and subsequent alkalization results in a cleavage product (P). 2B, 2C) Single (2B) and double-stranded (2C) oligonucleotides with the sequences given below were treated with the indicated concentrations of SsdA.

FIGS. 3A-3C. Proof of concept studies indicate DddA preferentially acts on unmethylated cytosines in DNA from mammalian cells. 3A) Relative number of the indication mutations detected in HeLa cell DNA treated with DddA or a 1:100 dilution of the DddA preparation (untreated). 3B) Sequence logos indicating relative frequencies of nucleotides in relationship to cytosines mutated to thymidine in HeLa cell DNA treated with DddA (top) or a 1:100 dilution of the DddA preparation (bottom). 3C) Frequency of the indicated mutations observed in DddA treated HeLa cell DNA, either pretreated with 5-azacytidine (aza) to prevent methylation or untreated, separated by methylation status as predicted by whole genome bisulfite conversion treatment and sequencing (WGBS).

FIG. 4. Median C•G-to-T•A conversion frequency across all 5′-TC-3′ positions of the E. coli genome as measured by whole genome sequencing of DNA treated with various doses of DddA (0.15 nM (0.005× of preparation) top panel, 1.5 nM (0.05× of preparation) middle panel, 15 nM (0.5× of preparation) bottom panel) with and without prior methylation. Conversion frequencies are stratified by sequence trimer 5′-TCN-3′ surrounding the deaminated C (left to right). Data from both unmethylated (light gray bars) and in vitro methylated (dark gray bars) using non-specific methyltransferase M.SssI acting at all 5′-CpG-3′ are shown. Trimer 5′-TCG-3′ where methylation occurs is boxed. Reduction of the C•G-to-T•A conversion frequency is maximal (5-fold) at intermediate doses of DddA treatment.

FIG. 5. Refined sequence context preference for enzyme DddA. The heatmap shows the per position weights of different base identities relative to the edited C towards DddA activity (in a context with fixed 5′-TC-3′ at positions −1 and 0). For example, a C at position −4 or a T at position+1 decrease DddA's activity, whereas an A at position −2 or a C at position+1 increase DddA's activity. Per position weights are the result of training a linear mathematical model which estimates conversion frequencies from any input DNA sequence contexts. Boxed weights were significant (three standard deviation) compared to models trained on shuffled sequences. Despite its low number of parameters, the model is predictive (Pearson correlation between observed and predicted 0.75), suggesting the per position activity weights above reflects DddA's bona fide quantitative sequence specificity.

### DETAILED DESCRIPTION

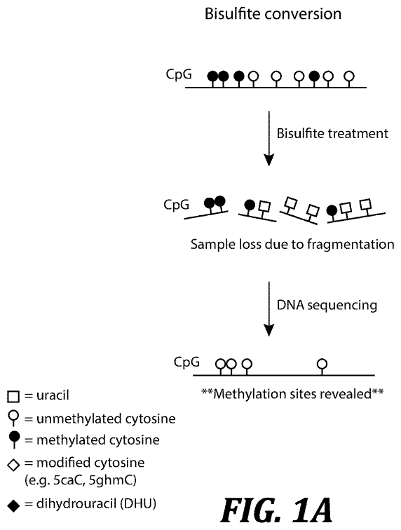

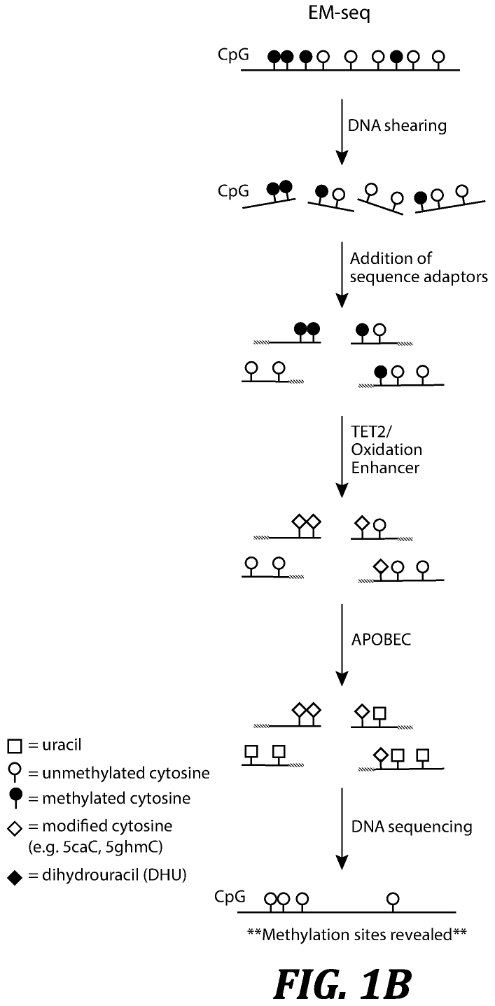

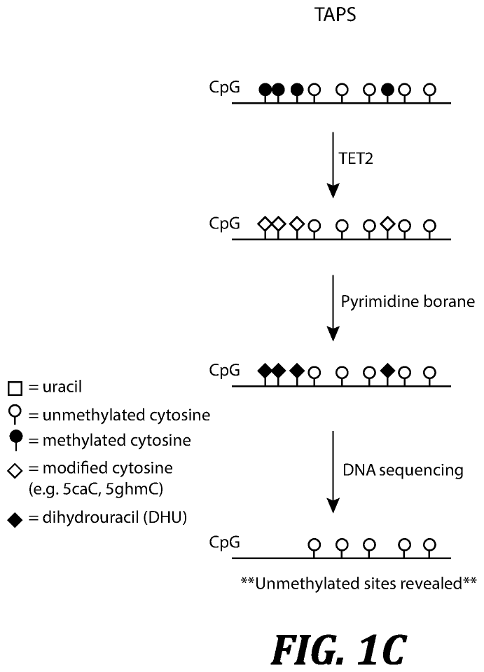

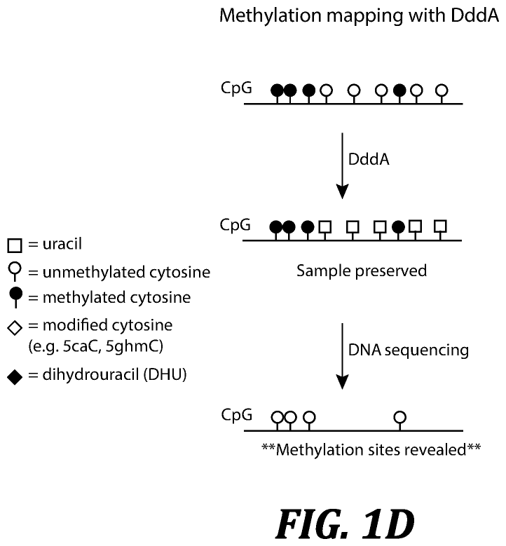

Methylation of cytosine residues in DNA is an important component of epigenetic gene regulation in many eukaryotic organisms and has been shown to be a key diagnostic biomarker for a number of cancers (see Kim, H., et al. (2018). Developing DNA methylation-based diagnostic biomarkers. J Genet Genomics 45, 87-97). However, the limitations of current technologies available for detecting sites of cytosine methylation in DNA have prevented the widespread implementation of methylation-based diagnostics (FIGS. 1A-1C). The most commonly employed method for detecting cytosine methylations involves treatment with bisulfite to convert methylated cytosine into uracil, which leads to the introduction of C•G-to-T•A transitions mutations upon PCR amplification and sequencing (FIG. 1A). A major disadvantage of this method is the harsh chemical nature of the bisulfite conversion treatment leads to significant DNA fragmentation and degradation and consequent loss of signal. Recently, a protocol which circumvents this problem by using the single-stranded cytosine deaminase APOBEC3a to convert unmethylated cytosine to uracil was developed (FIG. 1B) (see Vaisvila, R., et al. (2020). EM-seq: Detection of DNA Methylation at Single Base Resolution from Picograms of DNA. bioRxiv). However, this method, termed EM-seq, requires pretreatment of the DNA with TET2 and an Oxidation Enhancer to oxidize methylated cytosine into 5-carboxylcytosine, to protect them from deamination by APOBEC3a. Furthermore, EM-seq requires denaturation to generate single-stranded DNA. Another recently described approach for methylated cytosine mapping that circumvents the problem of harsh chemical treatment is TET-assisted pyridine borane sequencing (TAPS, FIG. 1C) [Liu, Y., et al. (2019). Bisulfite-free direct detection of 5-methylcytosine and 5-hydroxymethylcytosine at base resolution. Nat. Biotechnol. 37, 424-429]. In this method, methylated cytosine is oxidized by TET as in the EM-seq approach, followed by pyridine borane treatment to convert 5-carboxylcytosine to dihydrouracil (DHU). Like uracil, DHU residues in DNA are base paired with adenine by polymerase, so C•G-to-T•A transitions following amplification and sequencing can be used as a readout for methylated cytosines in this approach. TAPS performed better than bisulfite conversion at the whole genome level [Liu, Y., et al. (2019). Bisulfite-free direct detection of 5-methylcytosine and 5-hydroxymethylcytosine at base resolution. Nat. Biotechnol. 37, 424-429]. However, like EM-seq, it requires multiple DNA treatments prior to sequencing, which can limit its adaptation for diagnostic applications. Finally, nanopore sequencing platforms have been employed for the direct detection of modified bases in DNA [Rand, A. C., et al. (2017). Mapping DNA methylation with high-throughput nanopore sequencing. Nat Methods 14, 411-413]. This approach requires access to specialized equipment not yet widely available. Additionally, direct methylation detection methods are not currently amenable for diagnostic applications, as they cannot be targeted to specific sites of interest.

The present disclosure is based on the inventors' investigation into alternative methods to detect methylation events in nucleotide residues. As described in more detail below, the inventors demonstrated that multiple bacterial deaminases, namely active fragments of double-stranded DNA deaminase toxin A (DddA) and single-stranded DNA deaminase toxin A (SsdA), are able to selectively deaminate unmethylated cytosines. After simple treatment protocols using the bacterial deaminases, the resulting modified nucleic acid template can be sequenced using standard sequencing platforms without requiring specialized treatments or equipment, thus, providing a facile approach to determine the methylation status of residues in DNA.

In accordance with the foregoing, in one aspect the disclosure provides a method of deaminating one or more unmethylated cytosine residues in a polynucleic acid molecule. The method comprises contacting the polynucleic acid molecule with a bacterial cytosine deaminase. The contacting the polynucleic acid molecule with a bacterial cytosine deaminase can occur under standard enzymatic reaction conditions, including standard buffers, salts, etc., which are familiar in the art. Exemplary reaction conditions are discussed in more detail below.

In some embodiments, the bacterial cytosine deaminase selectively deaminates unmethylated cytosine residues. As used herein, the term “selectively deaminates” refers to the ability to significantly favor unmethylated cytosine residues for deamination over methylated cytosine residues. In some embodiments, the bacterial cytosine deaminase selectively deaminates unmethylated cytosine residues at a rate of at least 2×, 3×, 5×, 10×, 15×, 20×, 25×, 30×, 35×, 40×, 45×, 50×, 75×, 100×, 150×, 200×, 250×, 500× or more than the rate of deaminating the unmethylated cytosine residues. In some bacterial cytosine deaminase does not detectably deaminate methylated cytosines in the polynucleic acid under standard conditions.

In some embodiments, the bacterial cytosine deaminase is DddA, or a functional fragment or derivative thereof. In some embodiments, the DddA is from Burkholderia sp., such as a Burkholderia cenocepacia DddA, or a functional homolog thereof. A functional homolog is any DddA from other bacterial species with common evolutionary origin that retains the same core functional characteristics, namely possessing the ability to selectively deaminate unmethylated cytosine residues. The DddA can be obtained or derived from any bacterial source that has a functional homolog of DddA.

It is demonstrated below that the entire, full-length DddA enzyme is not required for functionality. For example, it was shown that a fragment of DddA with only the toxin domain was possessed selective deaminase functionality. A representative DddA (or functional fragment) comprises the amino acid sequence SEQ ID NO:1. Accordingly, the disclosure encompasses functional fragments of a DddA. For example, a functional fragment of a DddA can comprise an amino acid sequence with at least about 130 (e.g., about 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, and 164) contiguous amino acids of SEQ ID NO:1 or an amino acid sequence with at least about 80% (e.g., about 81%, 82%, 83%, 84%, 85%, 86%, 87%, 88%, 89%, 90%, 91%, 92%, 93%, 94%, 95%, 96%, 97%, 98%, 99%, and 100%) identity to at least about 130 contiguous amino acids (as described above) of SEQ ID NO:1. In some embodiments, the functional derivative of the DddA comprises an amino acid sequence with at least about 80%, at least about 85%, at least about 90%, at least about 95%, or at least about 98% to the amino acid sequence of SEQ ID NO:1.

In some reaction conditions, the concentration of the DddA or functional fragment or derivative thereof, can influence the selective deaminase functionality of the DddA. For example, it was shown that the DddA fragment comprising SEQ ID NO:1 had superior deaminase functionality at a medium concentration of approximately 1.5 nM. Thus, in some embodiments, the DddA or functional fragment or derivative thereof is contacted to the polynucleic acid molecule in a reaction where the functional fragment or derivative thereof is present at a concentration of about 0.5 nM to about 10 nM, such as about 0.5 nM to about 9 nM, about 0.5 nM to about 8 nM, about 0.5 nM to about 7 nM, about 0.5 nM to about 6 nM, about 0.5 nM to about 5 nM, about 0.5 nM to about 4 nM, about 0.5 nM to about 3 nM, about 0.5 nM to about 2 nM, about 0.75 nM to about 10 nM, about 0.75 nM to about 9 nM, about 0.75 nM to about 8 nM, about 0.75 nM to about 7 nM, about 0.75 nM to about 6 nM, about 0.75 nM to about 5 nM, about 0.75 nM to about 4 nM, about 0.75 nM to about 3 nM, about 0.75 nM to about 2 nM, about 1.0 nM to about nM, about 1.0 nM to about 9 nM, about 1.0 nM to about 8 nM, about 1.0 nM to about 7 nM, about 1.0 nM to about 6 nM, about 1.0 nM to about 5 nM, about 1.0 nM to about 4 nM, about 1.0 nM to about 3 nM, and about 1.0 nM to about 2 nM. In some embodiments, the DddA or functional fragment or derivative thereof is contacted to the polynucleic acid molecule in a reaction where the functional fragment or derivative thereof is present at a concentration of about 1.0 nM to about 2.0 nM, such as about 1.1 nM to about 1.9 nM, about 1.1 nM to about 1.9 nM, about 1.2 nM to about 1.8 nM, about 1.3 nM to about 1.7 nM, and about 1.4 nM to about 1.6 nM. In some embodiments, the DddA or functional fragment or derivative thereof is contacted to the polynucleic acid molecule in a reaction where the functional fragment or derivative thereof is present at a concentration of about 1.5 nM.

In some embodiments, the bacterial cytosine deaminase is SsdA, or a functional fragment or derivative thereof. In some embodiments, the SsdA is from a Pseudomonas sp., such as a Pseudomonas syringae SsdA, or a functional homolog thereof. A functional homolog is any SsdA from other bacterial species with common evolutionary origin that retains the same core functional characteristics, namely possessing the ability to selectively deaminate unmethylated cytosine residues. The SsdA can be obtained or derived from any bacterial source that has a functional homolog of SsdA.

It is demonstrated below that the entire, full-length SsdA enzyme is not required for functionality. For example, it was shown that a fragment of SsdA with only the toxin domain was possessed selective deaminase functionality. A representative SsdA (or functional fragment) comprises the amino acid sequence SEQ ID NO:2. Accordingly, the disclosure encompasses functional fragments of a SsdA. For example, a functional fragment of a SsdA can comprise an amino acid sequence with at least about 130 (e.g., about 131, 132, 133, 134, 135, 136, 137, 138, 139, 140, 141, 142, 143, 144, 145, 146, 147, 148, 149, 150, and 151) contiguous amino acids of SEQ ID NO:2 or an amino acid sequence with at least about 80% (e.g., about 81%, 82%, 83%, 84%, 85%, 86%, 87%, 88%, 89%, 90%, 91%, 92%, 93%, 94%, 95%, 96%, 97%, 98%, 99%, and 100%) identity to at least about 130 contiguous amino acids (as described above) of SEQ ID NO:2. In some embodiments, the functional derivative of the SsdA comprises an amino acid sequence with at least about 80%, at least about 85%, at least about 90%, at least about 95%, or at least about 98% to the amino acid sequence of SEQ ID NO:2.

The present method applies to any polynucleotide. In some embodiments, the polynucleic acid is or comprises DNA, such as genomic or mitochondrial DNA.

The polynucleotide can be from any source without limitation. In many embodiments, the polynucleotide is present in a biological sample and is isolated or purified from the biological sample according to standard protocols, without limitation. Nucleic acid isolation and purification techniques are known in the art and are encompassed by the disclosure. The biological samples can contain cells, tissues, or liquids (e.g., blood or blood derivative such as plasma or serum, cerebral spinal fluids, urine, sputum, etc.) waste. The biological sample can be an environmental sample. The biological sample can be obtained from an organism, such as a mammal (including humans, dogs, cats, rat, mouse, guinea pig, hamster, and mammals of agricultural interest), reptile, fish, bird, plant, etc.

In some embodiments, deamination of the one or more cytosine residues in the polynucleic acid molecule results in a cytosine to uracil conversion at the one or more cytosine residue positions to provide a modified polynucleic acid molecule (e.g., DNA) that contains one or uracil residues representing prior unmethylated cytosine residues as opposed to methylated cytosine residues. With the presence of the uracils, the modified polynucleotide can be sequenced using any appropriate sequencing platform that will distinguish the uracils. Thus, the method can further comprise detecting the presence of the uracil in the modified polynucleic acid. This detection can comprise performing sequence analysis, according to any standard sequencing method or using any acceptable sequencing platform, after contacting the polynucleotide with the bacterial cytosine deaminase.

In many embodiments the sequencing procedure includes initial amplification steps, e.g., using the polymerase chain reaction (PCR). For example, in PCR driven amplification, the uracils will be converted to thymine residues and, thus, will be sequenced as a thymine (T). Alternatively, the reverse complement strand will indicate an adenine (A) residue. Thus, the detection process comprises detecting introduction CG-to-TA transitions in the polynucleic acid. The transition can be determined by comparison to a known sequence. The known sequence can be derived or obtained from the same polynucleotide (or a molecule comprising the same polynucleotide), but which has not been exposed to a deaminase enzyme and, thus, provides an unmodified reference sequence. The reference polynucleic acid can be obtained from the same or similar biological sample as the polynucleic acid molecule contacted with the bacterial cytosine deaminase. In some embodiments, the method comprises generating the reference sequence. A C•G-to-T•A transition ultimately indicates the lack of methylation of the initial cytosine residue in the (pre-modified) polynucleic acid, whereas lack of C•G-to-T•A transition indicates methylated state of the initial cytosine residue in the (pre-modified) polynucleic acid.

Alternatively, the detection step can comprise other methods for the detection of nucleotide sequence variation, such as quantitative PCR, and other methods known in the art.

In another aspect, the disclosure provides a method of mapping methylated cytosine residues in a polynucleic acid molecule. The method comprises:
contacting a target polynucleic acid molecule with a bacterial cytosine deaminase for a sufficient time to deaminate unmethylated cytosine residues in the polynucleic acid molecule to provide a treated polynucleic acid molecule;
sequencing the treated polynucleic acid molecule to provide a treated sequence;
comparing the treated sequence to a reference sequence obtained from a reference polynucleic acid molecule identical to the target polynucleic acid molecule, wherein the reference polynucleic acid molecule is not contacted with a bacterial cytosine deaminase;
detecting introduction of one or more C•G-to-T•A transitions in the treated sequence compared to the reference sequence;
wherein the one or more C•G-to-T•A transitions correspond to unmethylated cytosine residues in the target polynucleotide and/or cytosine residues in the treated sequence correspond to methylated cytosine residues in the target polynucleotide.

In some embodiments, the bacterial cytosine deaminase is a DddA or functional fragment or derivative of DddA, as described in more detail above. In some embodiments, the DddA or functional fragment or derivative thereof is contacted to the polynucleic acid molecule in a reaction where the functional fragment or derivative thereof is present at a concentration of about 0.5 nM to about 10 nM, as described in more detail above. In some embodiments, the DddA or functional fragment or derivative thereof is contacted to the polynucleic acid molecule in a reaction where the functional fragment or derivative thereof is present at a concentration of about 1.0 nM to about 2.0 nM, such as about 1.1 nM to about 1.9 nM, about 1.1 nM to about 1.9 nM, about 1.2 nM to about 1.8 nM, about 1.3 nM to about 1.7 nM, and about 1.4 nM to about 1.6 nM. In some embodiments, the DddA or functional fragment or derivative thereof is contacted to the polynucleic acid molecule in a reaction where the functional fragment or derivative thereof is present at a concentration of about 1.5 nM.

In some embodiments, the bacterial cytosine deaminase is a SsdA or functional fragment or derivative of SsdA, as described in more detail above.

The method also applies to any polynucleotide. In some embodiments, the polynucleic acid is or comprises DNA, such as genomic or mitochondrial DNA.

As described above, the polynucleotide can be from any source without limitation. In many embodiments, the polynucleotide is present in a biological sample and is isolated or purified from the biological sample according to standard protocols, without limitation. Nucleic acid isolation and purification techniques are known in the art and are encompassed by the disclosure. The biological samples can contain cells, tissues, or liquids (e.g., blood or blood derivative such as plasma or serum, cerebral spinal fluids, urine, sputum, etc.) waste. The biological sample can be an environmental sample. The biological sample can be obtained from an organism, such as a mammal (including humans, dogs, cats, rat, mouse, guinea pig, hamster, and mammals of agricultural interest), reptile, fish, bird, plant, etc.

The methods of the disclosure can be further integrated into methods of diagnosis and/or treatment of diseases, e.g., some cancers, which are associated with methylation status of cytosine residues. For example, a biological sample can be obtained from a subject with a suspected disease or condition associated with a known cytosine methylation states or pattern of cytosine methylations. DNA is extracted from the biological sample and the method described above is deployed to determine the methylation status of cytosines in the subject's DNA. This status can then be used to determine the subject's status for the disease or condition and treatment can then be applied appropriately.

In another aspect, the disclosure provides a kit comprising a bacterial cytosine deaminase and reagents configured to facilitate deamination of cytosine residues in a polynucleic acid. The bacterial cytosine deaminase can be, e.g., DddA or SsdA, or a functional fragment or derivative thereof, as described above. The reagents configured to facilitate deamination can comprise one or more of buffers, salts, and the like. In some embodiments, the kit comprises a deamination buffer solution. An exemplary deamination buffer can include reagents such as NaCl, MES, DTT, and/or Ficoll PM70, in proportions that are configured to facilitate the deamination reaction. For example, the buffer reagents can be configured in the kit such that they are diluted to provide reaction conditions comprising: 75 mM NaCl, 20 mM MES pH 6.4, 2 mM DTT, and 8% w/v Ficoll PM70.

Generally, instructions comprise a description of administration or instructions for performance of an assay, such as the methods described above. The containers can be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits are provided in suitable packaging. Suitable packaging includes, but is not limited to, vials, bottles, jars, flexible packaging (e.g., sealed Mylar or plastic bags), and the like. A kit, or containers provided therein, can have a sterile access port (e.g. the container can be an intravenous solution bag or a vial having a stopper pierceable by a hypodermic injection needle). Kits can optionally provide additional components such as buffers and interpretive information. Normally, the kit comprises a container and a label or package insert(s) on or associated with the container.

### Additional Definitions

Unless specifically defined herein, all terms used herein have the same meaning as they would to one skilled in the art of the present invention. Practitioners are particularly directed to Sambrook J., et al. (eds.), Molecular Cloning: A Laboratory Manual, 3rd ed., Cold Spring Harbor Press, Plainsview, New York (2001); Ausubel, F. M., et al. (eds.), Current Protocols in Molecular Biology, John Wiley & Sons, New York (2010); Mirzaei, H. and Carrasco, M. (eds.), Modern Proteomics—Sample Preparation, Analysis and Practical Applications in Advances in Experimental Medicine and Biology, Springer International Publishing, 2016; and Comai, L., et al., (eds.), Proteomic: Methods and Protocols in Methods in Molecular Biology, Springer International Publishing, 2017, for definitions and terms of art.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

As used herein, the term “polypeptide” or “protein” refers to a polymer in which the monomers are amino acid residues that are joined together through amide bonds. When the amino acids are alpha-amino acids, either the L-optical isomer or the D-optical isomer can be used, the L-isomers being preferred. The term polypeptide or protein as used herein encompasses any amino acid sequence and includes modified sequences such as glycoproteins. The term polypeptide is specifically intended to cover naturally occurring proteins, as well as those that are recombinantly or synthetically produced.

One of skill will recognize that individual substitutions, deletions or additions to a peptide, polypeptide, or protein sequence which alters, adds or deletes a single amino acid or a percentage of amino acids in the sequence is a “conservatively modified variant” where the alteration results in the substitution of an amino acid with a chemically similar amino acid. Conservative amino acid substitution tables providing functionally similar amino acids are well known to one of ordinary skill in the art. The following six groups are examples of amino acids that are considered to be conservative substitutions for one another:
(1) Alanine (A), Serine (S), Threonine (T),
(2) Aspartic acid (D), Glutamic acid (E),
(3) Asparagine (N), Glutamine (Q),
(4) Arginine (R), Lysine (K),
(5) Isoleucine (I), Leucine (L), Methionine (M), Valine (V), and
(6) Phenylalanine (F), Tyrosine (Y), Tryptophan (W).

As used herein, the terms “nucleic acid” or “polynucleic acid” refer to a polymer of nucleotide monomer units or “residues”. The nucleotide monomer subunits, or residues, of the nucleic acids each contain a nitrogenous base (i.e., nucleobase) a five-carbon sugar, and a phosphate group. The identity of each residue is typically indicated herein with reference to the identity of the nucleobase (or nitrogenous base) structure of each residue. Canonical nucleobases include adenine (A), guanine (G), thymine (T), uracil (U) (in RNA instead of thymine (T) residues) and cytosine (C). However, the nucleic acids of the present disclosure can include any modified nucleobase, nucleobase analogs, and/or non-canonical nucleobase, as are well-known in the art. Modifications to the nucleic acid monomers, or residues, encompass any chemical change in the structure of the nucleic acid monomer, or residue, which results in a noncanonical subunit structure. Such chemical changes can result from, for example, epigenetic modifications (such as to genomic DNA or RNA), or damage resulting from radiation, chemical, or other means. Illustrative and nonlimiting examples of noncanonical subunits, which can result from a modification, include uracil (for DNA), 5-methylcytosine, 5-hydroxymethylcytosine, 5-formethylcytosine, 5-carboxycytosine b-glucosyl-5-hydroxy-methylcytosine, 8-oxoguanine, 2-amino-adenosine, 2-amino-deoxyadenosine, 2-thiothymidine, pyrrolo-pyrimidine, 2-thiocytidine, or an abasic lesion. An abasic lesion is a location along the deoxyribose backbone but lacking a base. Known analogs of natural nucleotides hybridize to nucleic acids in a manner similar to naturally occurring nucleotides, such as peptide nucleic acids (PNAs) and phosphorothioate DNA.

Reference to sequence identity addresses the degree of similarity of two polymeric sequences, such as nucleic acid or protein sequences. Determination of sequence identity can be readily accomplished by persons of ordinary skill in the art using accepted algorithms and/or techniques. Sequence identity is typically determined by comparing two optimally aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. Various software driven algorithms are readily available, such as BLAST N or BLAST P to perform such comparisons.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed.

### Example 1

The following describes studies demonstrating use of bacterial deaminases to differentiate and detect methylation events on cytosine residues.

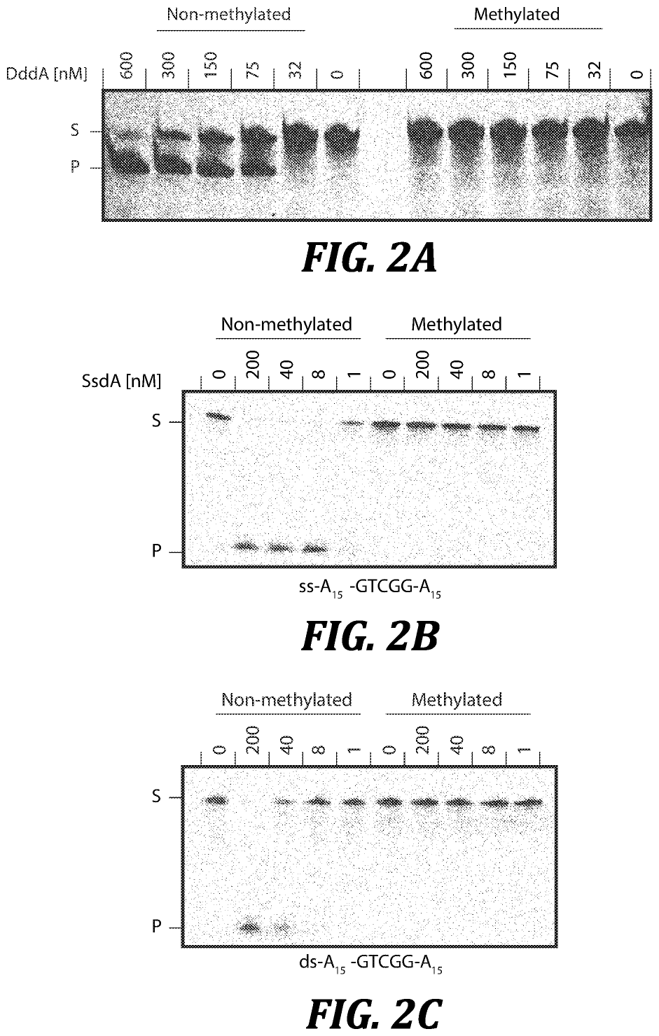

The inventors have developed a simple, easy to implement method for the detection of methylated cytosines that capitalizes on the DNA cytosine deaminase activity of DddA and other bacterial cytosine deaminases (FIG. 1D). Experiments described herein demonstrate that, unlike APOBEC3a (Schutsky, E. K., et al. (2017). APOBEC3A efficiently deaminates methylated, but not TET-oxidized, cytosine bases in DNA. Nucleic Acids Res. 45, 7655-7665), the activity of two divergent bacterial cytidine deaminases with differing substrate contexts is strongly inhibited by cytosine methylation (FIGS. 2A-2C). The effect of cytosine methylation on deamination activity was demonstrated using in vitro deamination assays. For each assay, 1 μM of a 5′-FAM labeled DNA oligonucleotide probe (5′-FAM-A(14)-GCTCGGA-A(14)-3′), the sequence of which is set forth in SEQ ID NO:3, containing either methylated or unmethylated cytosine was mixed with deamination buffer (DddA: 20 mM MES pH 6.4, 75 mM NaCl, 2 mM DTT, 8% Ficoll 70, SsdA: 75 mM NaCl, 20 mM Tris-HCl pH 7.4, 2 mM DTT) and a range of concentrations of the purified enzyme toxins domains (see FIGS. 2A-2C) and incubated for 1 hr at 37° C. For SsdA, both single- and double-stranded substrates were employed, while only double-stranded substrate was used for DddA. Reactions were then stopped by adding Udg solution (New England Biolabs, 0.02 U μl-1 UDG in 1×UDG buffer) and further incubated for 30 min. Cleavage of substrates was induced by addition of 100 mM NaOH and incubation at 95° C. for 3 min. Samples were then analyzed by denaturing 15% acrylamide gel electrophoresis and the resulting fluorescent DNA fragments were detected by fluorescence imaging with Azure Biosystems. A shift in the size of the DNA fragment provides evidence of cytosine deamination.

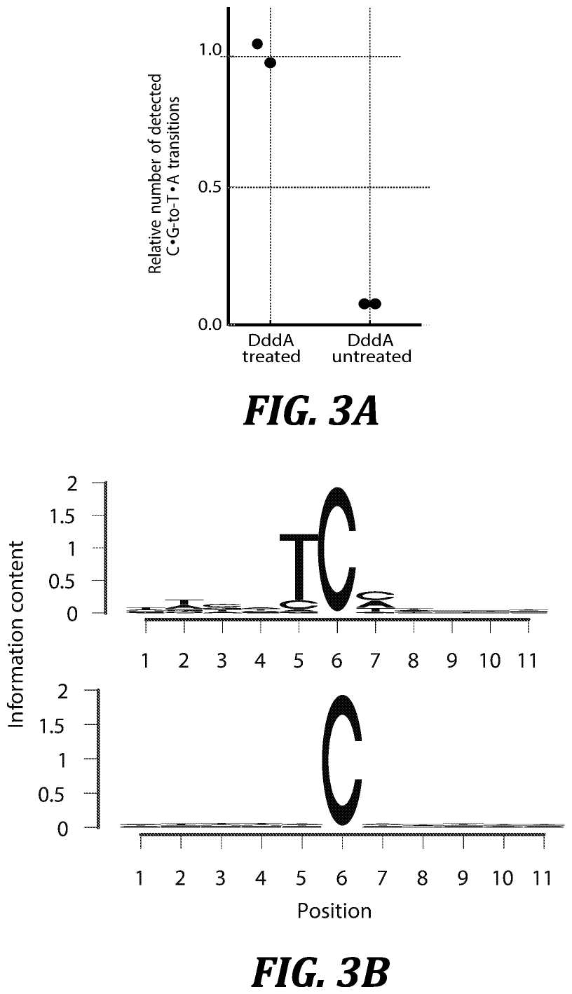

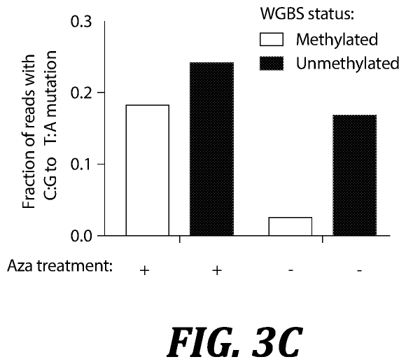

With more complex templates than the purified oligonucleotides described above, the activity of cytosine deaminases can be detected by sequencing, as they catalyze cytosine to uracil conversions, which result in C to T transition mutations. In an initial proof-of-concept experiment for the use of bacterial cytosine deaminase enzymes for methylation-mapping on a genome scale, the inventors assessed the sensitivity of cytosine deaminase DddA to the methylation state of human DNA, as determined previously through whole genome bisulfite conversion treatment and sequencing (WGBS) [Lee, D., et al. (2020). Epigenome-based splicing prediction using a recurrent neural network. PLoS Comput. Biol. 16, e1008006]. To do this, 100 ng of genomic DNA from cultured HeLa cells (purified from DNeasy kit, Qiagen, following manufacturer's instructions) was treated with a purified 0.17 nM preparation of the active domain of DddA prepared in-house from cloned dddA expressed in E. coli (comprising an amino acid sequence as set forth in SEQ ID NO:1) for one hour (in deamination buffer: final concentrations 75 mM NaCl, 20 mM MES pH 6.4, 2 mM DTT, 8% w/v Ficoll PM70, 1 h treatment at 37 C). The reaction was cleaned up (Zymo Clean & Concentrator) and prepared for sequencing library generation (acoustic shearing with Covaris to target size 150 bp, AMPure XP clean up, library preparation using Illumina Truseq DNA sample preparation kit following manufacturer's protocol [end-repair, A-tailing, ligation with indexed Y-adapters] with the exception that the final PCR was performed with uracil tolerant polymerase [KAPA HiFi Uracil+, Roche]). Subsequent Illumina-based whole-genome sequencing revealed an over 10-fold increase in the number of detected C•G-to-T•A transitions compared to 100-fold diluted DddA treatment controls (FIG. 3A). Detected C•G-to-T•A transitions occurred preferentially in a 5′-TC-3′ context, as expected from the known substrate preference of DddA (FIG. 3B, top). This 5′-TC-3′ enrichment was not observed for the C•G-to-T•A transitions detected from DNA treated with a 1:100 dilution of the DddA preparation (FIG. 3B, bottom). These results establish the enzymatic activity of DddA on human genomic DNA. Importantly, this activity was shown to be sensitive to the methylation state of DNA: a nearly 10-fold increase in the frequency of C•G-to-T•A transitions at 5′-TCG-3′ sites with unmethylated cytosine was observed (as determined from WGBS data in HeLa cells [Lee, D., et al. (2020). Epigenome-based splicing prediction using a recurrent neural network. PLoS Comput. Biol. 16, e1008006]) compared to sites with methylated cytosines (FIG. 3C). Pretreatment of HeLa cells with 5-azacytidine to block methylation prior to genomic DNA extraction and DddA treatment largely eliminated this difference. These preliminary results strongly support the utility of using bacterial cytosine deaminases for mapping DNA methylation. Importantly, the method can flexibly operate in a shotgun manner or at selected loci, the latter simply by coupling it to well-established methods for targeted enrichment (e.g. PCR, hybrid capture, etc.).

To further characterize the sequence specificity and dose dependence of the enzymatic activity of DddA, bacterial genomic DNA from Escherichia coli was treated at various doses of DddA. Bacterial genomic DNA was selected as a template to enable high sequencing coverage at moderate cost while retaining high diversity of sequence context to test DddA's activity. Importantly, purified E. coli DNA (40 ng/μL in a 50 μL reaction) was either treated with methyltransferase M.SssI (NEB, following manufacturer's protocol: in 50 uL: 1× Methyltransferase buffer, 0.64 mM SAM, 16 units M.SssI. Treatment was carried out for 4 h at 37 C followed by 5 min 65 C heat inactivation), which methylates all cytosines in a 5′-CpG-3′ context (in vitro methylated), or left untreated (non-methylated), providing an ideal template to validate the methylation dependence of DddA. Following purification by isopropanol precipitation, 100 ng of E. coli DNA was subjected to DddA treatment (in the same deamination buffer as above) at various concentrations in 12 μL reactions (0.15 nM, 1.5 nM, and 15 nM of the enzyme preparation, 1 h at 37 C). Subsequent to DddA treatment, DNA was purified by isopropanol precipitation and prepared for sequencing library generation (tagmentation using Illumina Nextera XT, amplification using uracil tolerant polymerase [KAPA HiFi Uracil+, Roche]). The resulting Illumina based whole-genome sequencing data was analyzed to calculate the rate of C•G-to-T•A conversions. High coverage on the genome permitted calculation of the conversion frequency (fraction of sequencing reads supporting the converted allele over all reads covering that position) at all genomic positions, yielding quantitative information on DddA's activity in a broad range of sequence contexts.

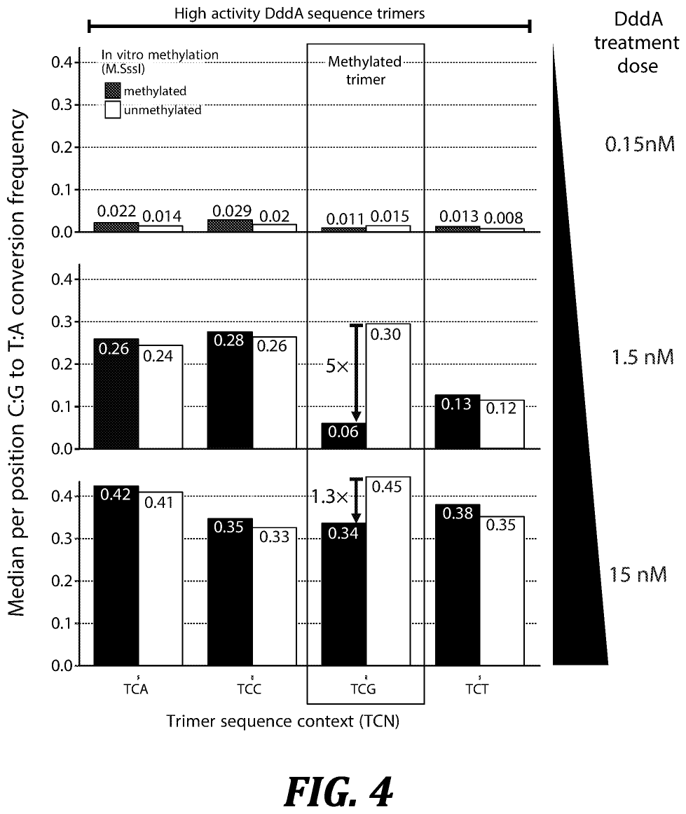

In support of the results on HeLa cell DNA, DddA-induced C•G-to-T•A conversions in the 5′-TC-3′ contexts were strongly dependent on methylation status. Importantly, titrating the DddA dose revealed that an intermediate DddA dose of 1.5 nM led to a maximum difference in conversion frequencies between the methylated and unmethylated samples (5-fold reduction in median conversion frequency in methylated vs. unmethylated sample, FIG. 4 middle panel). At low DddA doses, little conversions were observed irrespective of methylation status. Conversely, at high DddA doses, while reduction in C•G-to-T•A conversions were still observed in methylated contexts, the magnitude of the effect was substantially reduced compared to intermediate DddA doses (1.3-fold vs. 5-fold reduction, FIG. 4 bottom vs. middle panel). This suggests that DddA's lower activity at methylated Cs can be compensated by very high dose treatments, underscoring the need for optimization of the enzymatic treatment in a methylation detection assay. It is noted that the residual activity of DddA at dose 1.5 nM in the 5′-TCG-3′ context could reflect incomplete methylation in vitro by enzyme M.SssI, as opposed to promiscuous DddA activity on methylated substrate. In support of this, bisulfate treatment of M.SssI-treated E. coli DNA did reveal small fraction of residual unmethylated corresponding possibly corresponding to a substantial fraction of the C•G-to-T•A conversions in the 0.05× DddA dose in the 5′-TCG-3′ methylated sample (not shown).

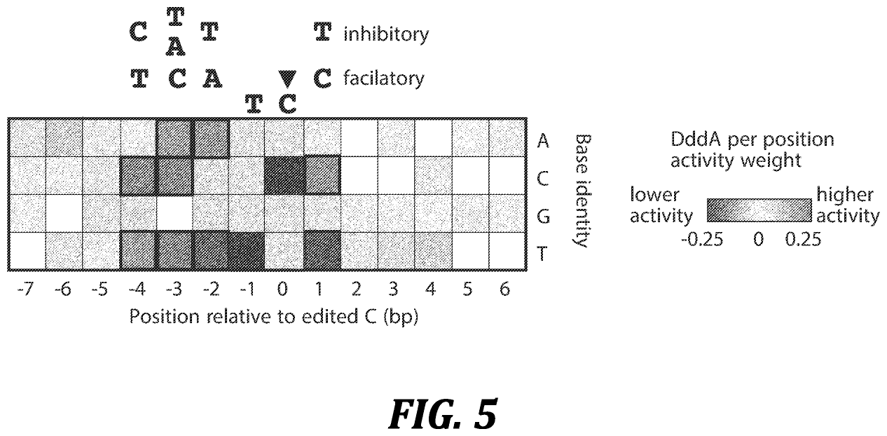

Next, the high coverage of the dataset was leveraged to gain refined information about the sequence specificity of DddA. Following the protocol disclosed in Zhang et al, Searching for sequence features that control DNA flexibility, arXiv:2012.06127, the data was used to train a mathematical model that linearly weighs the base identity at each position in the vicinity of the edited C. The specificity model takes as input any sequence of interest (surrounding core 5′-TC-3′), and yields as output predicted conversion frequency for the edited C. Despite having few parameters, the model predicted with high accuracy the measured conversion frequencies observed across sequence contexts which spanned a 100-fold range (Pearson correlation between predicted and observed 0.75, not shown). Trained weights in the model highlighted specific bases at positions relative to the deaminated C with either faciliatory or inhibitory effects on the activity of DddA. Sequence contexts with largest inhibitory effects are identified to be a C at position −4 (relative to the edited C), T or A at −3, T at −2, and T at position+1. Sequence contexts with largest faciliatory effects are identified to be a T at position −4, C at −3, A at −1, and C at +1. See, e.g., FIG. 5. This quantitative sequence specificity could further be leveraged to increase sensitivity to methylation detection within a DddA-based assay.

This date demonstrates that bacterial cytosine deaminases such as DddA and homologs and minor variants thereof are useful for selective conversion of unmethylated cytosines in nucleic acids and can be applied broader analyses to map methylation. Such methods have utility for detection of diagnostic biomarkers for cancer and/or tissue damage, as well as for any other research or clinical application involving DNA methylation mapping.

While illustrative embodiments have been illustrated and described, it will be appreciated that various changes can be made therein without departing from the spirit and scope of the invention.

1. A method of deaminating one or more unmethylated cytosine residues in a polynucleic acid molecule, comprising contacting the polynucleic acid molecule with a bacterial cytosine deaminase, wherein the bacterial cytosine deaminase does not deaminate methylated cytosines in the polynucleic acid.

2. (canceled)

3. The method of claim 1, wherein the bacterial cytosine deaminase is double-stranded DNA deaminase toxin A (DddA), or a functional fragment or derivative thereof, and wherein the DddA or functional fragment or derivative of DddA comprises an amino acid sequence selected from:
(i) an amino acid sequence with at least 130 contiguous amino acids of SEQ ID NO:1 or an amino acid sequence with at least about 80% identity to 130 contiguous amino acids of SEQ ID NO:1; and
(ii) an amino acid sequence with at least about 80%, at least about 85%, at least about 90%, at least about 95%, or at least about 98% to the amino acid sequence of SEQ ID NO:1.

4-5. (canceled)

6. The method of claim 3, wherein the DddA or functional fragment or derivative thereof is contacted to the polynucleic acid molecule in a reaction, and wherein the functional fragment or derivative thereof is present at a concentration of about 0.5 nM to about 10 nM.

7. The method of claim 1, wherein the bacterial cytosine deaminase is single-stranded DNA deaminase toxin A (SsdA), or a functional fragment or derivative thereof, and wherein the SsdA or a functional fragment or derivative of SsdA comprises an amino acid sequence selected from:
(i) an amino acid sequence with at least 130 contiguous amino acids of SEQ ID NO:2 or an amino acid sequence with at least about 80% identity to 130 contiguous amino acids of SEQ ID NO:2; and
(ii) an amino acid sequence with at least about 80%, at least about 85%, at least about 90%, at least about 95%, or at least about 98% to the amino acid sequence of SEQ ID NO:2.

8-9. (canceled)

10. The method of claim 1, further comprising isolating or purifying the polynucleic acid from a biological sample, wherein the polynucleic acid is DNA, optionally wherein the DNA is genomic or mitochondrial DNA.

11-12. (canceled)

13. The method of claim 10, further comprising isolating the DNA from a cell or plurality of cells.

14. The method of claim 1, wherein deamination of the one or more cytosine residues in the polynucleic acid molecule results in a cytosine to uracil conversion.

15. The method of claim 14, further comprising detecting the occurrence of one or more deamination events in the polynucleic acid, wherein detecting the occurrence of the deamination event(s) in the polynucleic acid comprises sequencing the polynucleic acid after contacting with the bacterial cytosine deaminase and detecting introduction of one or more C•G-to-T•A transitions in the polynucleic acid.

16. (canceled)

17. The method of claim 15, wherein detecting introduction of one or more C•G-to-T•A transitions in the polynucleic acid comprises comparing the sequence of the polynucleic acid with a reference polynucleic acid sequence obtained from a reference polynucleic acid that has not been contacted with the bacterial cytosine deaminase, and wherein the reference polynucleic acid is obtained from the same or similar biological sample as the polynucleic acid molecule contacted with the bacterial cytosine deaminase.

18. (canceled)

19. A method of mapping methylated cytosine residues in a polynucleic acid molecule, comprising:
contacting a target polynucleic acid molecule with a bacterial cytosine deaminase for a sufficient time to deaminate unmethylated cytosine residues in the polynucleic acid molecule to provide a treated polynucleic acid molecule;
sequencing the treated polynucleic acid molecule to provide a treated sequence;
comparing the treated sequence to a reference sequence obtained from a reference polynucleic acid molecule identical to the target polynucleic acid molecule, wherein the reference polynucleic acid molecule is not contacted with a bacterial cytosine deaminase;
detecting introduction of one or more C•G-to-T•A transitions in the treated sequence compared to the reference sequence;
wherein the one or more C•G-to-T•A transitions correspond to unmethylated cytosine residues in the target polynucleotide and/or C residues in the treated sequence correspond to methylated cytosine residues in the target polynucleotide.

20. The method of claim 19, wherein the bacterial cytosine deaminase is double-stranded DNA deaminase toxin A (DddA), or a functional fragment or derivative thereof, wherein the DddA or functional fragment or derivative of DddA comprises an amino acid sequence selected from:
(i) an amino acid sequence with at least 130 contiguous amino acids of SEQ ID NO:1 or an amino acid sequence with at least about 80% identity to 130 contiguous amino acids of SEQ ID NO:1; and
(ii) an amino acid sequence with at least about 80%, at least about 85%, at least about 90%, at least about 95%, or at least about 98% to the amino acid sequence of SEQ ID NO:1.

21-22. (canceled)

23. The method of claim 20, wherein the DddA or functional fragment or derivative thereof is contacted to the polynucleic acid molecule in a reaction, and wherein the functional fragment or derivative thereof is present at a concentration of about 0.5 nM to about 10 nM.

24. The method of claim 19, wherein the bacterial cytosine deaminase is SsdA, or a functional fragment or derivative thereof, wherein the SsdA or a functional fragment or derivative of SsdA comprises an amino acid sequence selected from:
(i) an amino acid sequence with at least 130 contiguous amino acids of SEQ ID NO:2 or an amino acid sequence with at least about 80% identity to 130 contiguous amino acids of SEQ ID NO:2; and
ii) an amino acid sequence with at least about 80%, at least about 85%, at least about 90%, at least about 95%, or at least about 98% to the amino acid sequence of SEQ ID NO:2.

25-26. (canceled)

27. The method of claim 19, wherein the polynucleic acid is DNA, and optionally wherein the DNA is genomic or mitochondrial DNA.

28. (canceled)

29. The method of claim 19, further comprising isolating the DNA from a biological sample.

30. A kit comprising a bacterial cytosine deaminase and reagents configured to facilitate deamination of cytosine residues in a polynucleic acid.

31. The kit of claim 30, wherein the bacterial cytosine deaminase is DddA, or a functional fragment or derivative thereof, wherein the DddA or functional fragment or derivative of DddA comprises an amino acid sequence selected from:
(i) an amino acid sequence with at least 130 contiguous amino acids of SEQ ID NO:1 or an amino acid sequence with at least about 80% identity to 75 contiguous amino acids of SEQ ID NO:1; and
(ii) an amino acid sequence with at least about 80%, at least about 85%, at least about 90%, at least about 95%, or at least about 98% to the amino acid sequence of SEQ ID NO:1.

32-33. (canceled)

34. The kit of claim 30, wherein the bacterial cytosine deaminase is SsdA, or a functional fragment or derivative thereof, wherein the SsdA or a functional fragment or derivative of SsdA comprises an amino acid sequence selected from:
(i) an amino acid sequence with at least 130 contiguous amino acids of SEQ ID NO:2 or an amino acid sequence with at least about 80% identity to 75 contiguous amino acids of SEQ ID NO:2; and
(ii) an amino acid sequence with at least about 80%, at least about 85%, at least about 90%, at least about 95%, or at least about 98% to the amino acid sequence of SEQ ID NO:2.

35-36. (canceled)

37. The kit of claim 30, wherein the reagents are configured to facilitate deamination comprise one or more of buffers, salts, and the like, and wherein the reagents configured to facilitate deamination comprise a deamination buffer comprising NaCl, MES, DTT, and/or Ficoll PM70.

38. (canceled)
